(19) United States
(12) Patent Application Publication
(10) Pub. No.: US 2026/0294763 A1
(43) Pub. Date: Oct. 1, 2026

(54) COMPOSITIONS COMPRISING NIACINAMIDE AND RESVERATROL SALICYLATE

(71) Applicant: ELC Management LLC, Melville, NY (US)

(72) Inventors: Klodjan STAFA, Ardsley, NY (US); Earl C. GOYARTS, Commack, NY (US); Kelly DONG, Merrick, NY (US); Kelsey MORRIS, Freeport, NY (US); James McCARTHY, Babylon, NY (US); Thomas MAMMONE, Farmingdale, NY (US); Nadine PERNODET, Dix Hills, NY (US)

(21) Appl. No.: 19/480,816

(22) PCT Filed: Apr. 30, 2024
(86) PCT No.: PCT/US2024/027055

§ 371 Date: Nov. 2, 2025

Related U.S. Application Data
(60) Provisional application No. 63/499,565, filed on May 2, 2023.

(51) Int. Cl. A61K 8/37; A61K 8/06; A61K 8/41; A61K 8/60; A61K 8/67; A61Q 19/00

(52) U.S. Cl. CPC A61K 8/37; A61K 8/062; A61K 8/064; A61K 8/416; A61K 8/60; A61K 8/675; A61Q 19/00

(57) ABSTRACT

A topical skincare composition that comprises niacinamide and one or more resveratrol salicylates. The compositions increase the levels of energy in epidermal keratinocytes, while mitigating oxidative damage associate with increased ATP production due to application of niacinamide. Also increased is the expression of AQP-3, CLDN-1 and CLDN-4, leading to improved barrier function and increased moisture retention in skin. Preferred compositions are in the form of creams, lotions, serums or gels, implemented as mixtures or emulsions.

### FIELD OF THE INVENTION

The present invention is in the field of topical compositions, more specifically topical skincare compositions that boost energy levels and regulate moisture content in skin cells, while mitigating the effects of oxidation.

### BACKGROUND OF THE INVENTION

Adenosine monophosphate-activated protein kinases (AMPK) are considered a master regulator of metabolism, affecting numerous metabolic pathways. AMPK is an enzyme complex that detects the AMP/ATP ratio within cells. When this ratio increases (indicating energy depletion), the AMPK metabolic switch is activated, which leads to a number of measures to increases cellular energy levels, such as fat burning, ketosis, and increased glucose uptake by cells. If the energy deficit is maintained for long enough, AMPK triggers a process leading to mitochondrial biogenesis (creation of new mitochondria). Among known activators of AMPK are niacinamide and resveratrol.

Niacinamide (also known as nicotinamide) is one of three forms of Vitamin B3. It is water soluble, and has the following general formula.

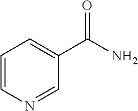

Niacinamide has been used both orally and topically. It is known to apply niacinamide topically to treat acne, psoriasis, rosacea and other inflammatory skin conditions. It has been suggested that niacinamide may display anti-inflammatory, anti-ageing, anticarcinogenic and skin moisturizing properties. See, for example, Chen, A. C. N., & Damian, D. L. (2014). Nicotinamide and the skin. Australasian Journal of Dermatology, 55 (3), 169-175. doi:/10.1111/ajd.12163.

Niacinamide is a precursor of nicotinamide adenine dinucleotide (NAD). Within cells, niacinamide is converted into nicotinamide adenine dinucleotide, which acts as an electron transporter, and is an essential component for energy production (ATP) in cells through the electron transport chain and oxidative phosphorylation. Increased availability of precursor niacinamide is thought to boost ATP production and enhance energy-dependent cellular processes such as DNA repair. At the same time, as cells make more energy, levels of potentially harmful byproducts, specifically reactive oxygen species, are also increased

Resveratrol, also referred to as 3,4′,5-trihydroxystilbene is a polyhydroxy-substituted compound having the general formula:

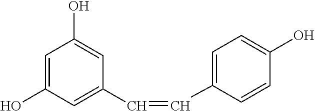

Resveratrol has strong antioxidant, anti-inflammatory and pro-longevity properties. It helps stimulate collagen production while protecting the skin from the damaging effects of free radicals. Because of its perceived fountain-of-youth properties, resveratrol has been incorporated into a variety of cosmetic formulations such skin creams. However, one problem with resveratrol is that it is generally unstable in cosmetic formulations. This issue has been addressed by derivatizing the resveratrol by reacting one or more of the hydroxyl groups with substituents that confer stability when the resveratrol is formulated into cosmetic products. In particular, it is known that esterifying resveratrol with inorganic acids, organic carboxylic acids, or reacting the hydroxyl groups to form alkoxy substituents or glycosides, provides resveratrol derivatives that are stable in cosmetic emulsions and enable formulation of cosmetic products with the aesthetics and stability that are necessary for commercially successful products. See, for example, U.S. Pat. Nos. 8,084,496 and 9,220,669, wherein salicylate esters of resveratrol are described.

### OBJECT OF THE INVENTION

A main object of the invention is to provide a topical composition that increases energy levels in skin cells by increasing the rate of ATP generation, while also providing protection from oxidative species that are created in the process.

Another object of the invention is to increase the levels of AQP-3, CLDN-1 & CLDN-4 in epidermal keratinocytes leading to improved barrier function and increased moisture retention in skin.

It is a further object of the invention to provide skin care compositions comprising niacinamide, at least one resveratrol salicylate, an aqueous phase and, optionally, an oil phase.

### SUMMARY OF THE INVENTION

The present invention is a topical skincare composition that comprises niacinamide and one or more resveratrol salicylates. Preferred compositions are in the form of creams, lotions, emulsions, gels, serums and/or powders.

### DESCRIPTION OF THE FIGURES

FIG. 1 shows the rate of consumption of oxygen by NHDF treated with niacinamide.

FIG. 2 shows the rate of consumption of oxygen by NHDF treated with niacinamide and resveratrol salicylate.

FIG. 3 shows the changes in levels of peroxide species in NHDF, comparing niacinamide, resveratrol salicylate, and a combination of niacinamide and resveratrol salicylate.

FIG. 4 shows the oxygen consumption rate by NHDF treated with niacinamide, over a period of ninety minutes.

FIG. 5 shows the oxygen consumption rate by NHDF treated with niacinamide and resveratrol salicylate, over a period of ninety minutes.

FIGS. 6 and 7 show measurements of NAD+/NADH in the 28yo and 68yo NHDF cells treated with various concentrations of resveratrol salicylate, niacinamide, and a combination of niacinamide and resveratrol salicylate

FIG. 8 shows changes in the ratio of phosphorylated (activated) AMPK to total AMPK in NHDF treated with a combination of niacinamide and resveratrol salicylate.

FIG. 9 shows the results of FIG. 8, expressed as a percentage of the control.

FIG. 10 shows the mass amount per mL of phosphorylated (activated) AMPK, measured by enzyme-linked immunosorbent assay (ELISA).

FIG. 11 shows the results of FIG. 10, expressed as a percentage of the control.

FIGS. 12A-C and 13A-C show changes in the expression of hydration targets genes, Aquaporin-3, Claudin-1 and Claudin-4, in 28 year old and 56 year old male NHEK, as a result of treatment with a combination of niacinamide and resveratrol salicylate.

### DETAILED DESCRIPTION

A topically applied skincare composition comprises a cosmetically acceptable base, niacinamide and one or more resveratrol salicylate esters. The base will generally be aqueous, wherein the amount of water may range from about 0.1-99%, preferably from about 5-85%, more preferably from about 7-75% by weight of the total composition. Some preferred embodiments of the present invention may comprise an oil phase. For example, the composition may be in the form of a water-in-oil or oil-in-water emulsion. The amount of oil in the oil phase will preferably range from about 1-95%, preferably from about 5-85%, more preferably from about 7-65% by weight of the total composition.

Resveratrol salicylate esters that may be useful in the present invention include: 3-salicylate-5,4′-dihydroxystilbene; 5-salicylate-3,4′-dihydroxystilbene; 4′-salicylate-3,5-dihydroxystilbene; 3,5-disalicylate-4′-hydroxystilbene; 3,4′-disalicylate-5-hydroxystilbene; 4′,5-disalicylate-3-hydroxystilbene; 3,5,4′-trisalicylate stilbene; or combinations thereof.

### Effects on ATP Production and Oxidative Damage

We have demonstrated that one or more resveratrol salicylate esters, when used in combination with niacinamide, mitigates the oxidative damage caused by increased ATP production in cells. The following experimental results demonstrate the usefulness of resveratrol salicylate in combination with niacinamide. Various measurements were made on 28 and 68 year old normal human dermal fibroblasts (NHDF).

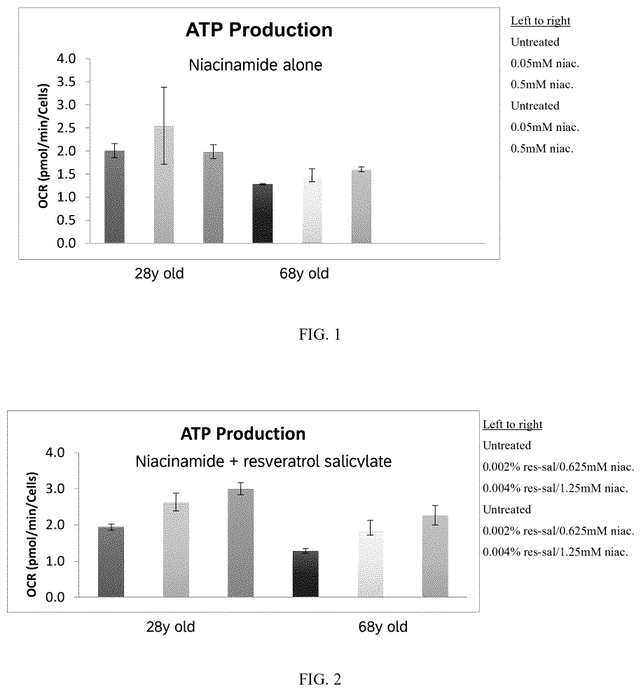

FIGS. 1 and 2 show the rate of consumption of oxygen by NHDF, indicating changes in levels of ATP production. The trend suggests an increase in available cellular energy.

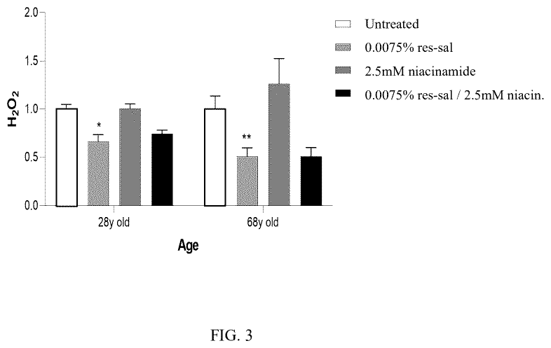

FIG. 3 shows the changes in levels of peroxide species in NHDF. The results have been normalized so that the level of H₂O₂ for untreated cells is 1. While niacinamide alone significantly increased the level of oxidative species the 68 year old cells, resveratrol salicylate erased all of that, and more. In the 28 year old cells, resveratrol salicylate reduced oxidation below the levels of untreated cells, despite the application of niacinamide.

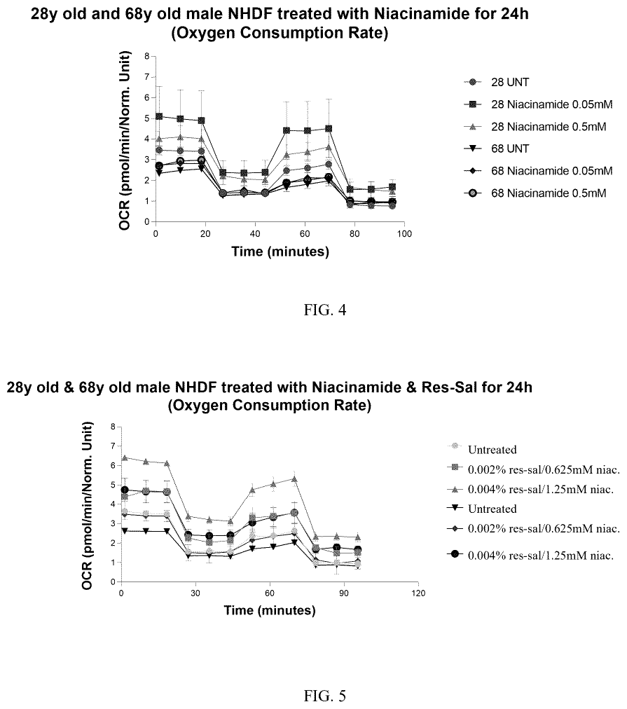

FIGS. 4 and 5 show the oxygen consumption rate by the NHDF over a period of ninety minutes. FIG. 4 shows cells treated with niacinamide; FIG. 5 shown cells treated with the combination of niacinamide and resveratrol salicylate. The data suggests that the combination of niacinamide and resveratrol salicylate tends to enhance ATP production more than niacinamide alone.

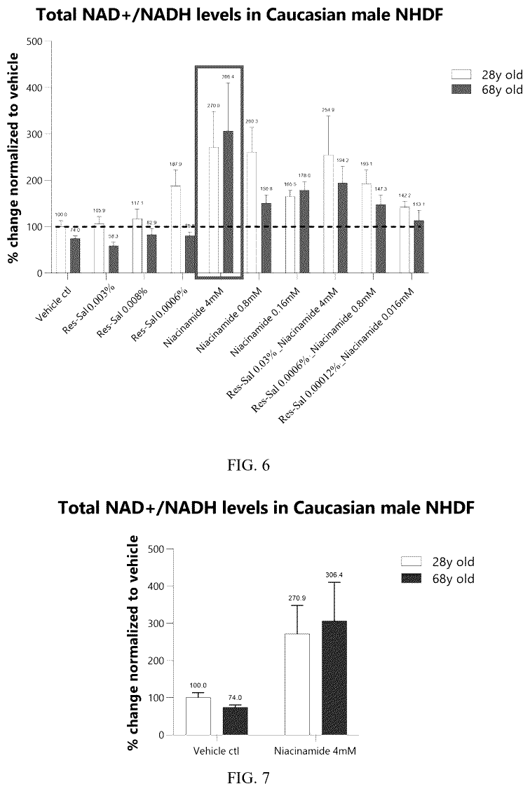

FIGS. 6 and 7 show measurements of NAD+/NADH in the 28yo and 68yo NHDF cells. Test samples were prepared at three different concentrations of resveratrol salicylate, three different concentrations of niacinamide, and three different concentrations the combination. Results indicate niacinamide increases the production of NADH in both cell lines.

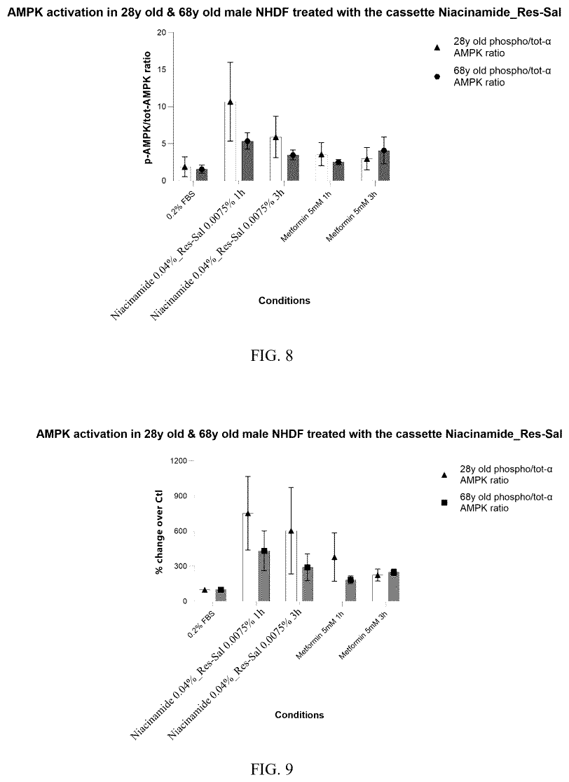

FIG. 8 shows changes in the ratio of phosphorylated (activated) AMPK to total AMPK in the 28yo and 68yo NHDF. Measurements were made at 1 hour and 3 hours after application. Metformin, a known activator of AMPK is included for comparison. FBS alone served as a control. Increases in the ratio indicate greater energy production. In FIG. 9, the results are expressed as a percentage of the control. The combination of niacinamide (0.04%) and resveratrol salicylate (0.1%) treatment for 1 hour and 3 hour in low serum media (e.g. 0.2% FBS) activates the AMPK pathway to generate more energy.

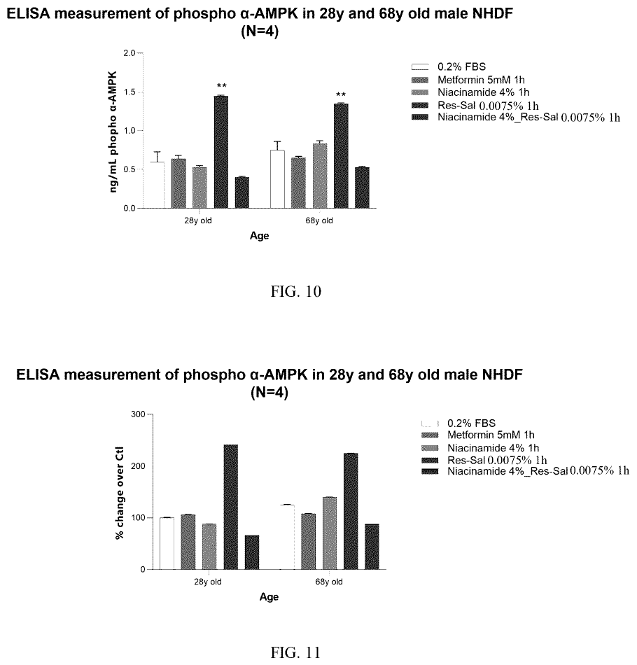

Levels of phosphorylated (activated) AMPK were also measured by enzyme-linked immunosorbent assay (ELISA). FIG. 10 shows the mass amount per mL, while FIG. 11 shows the results as a percentage of the control. Measurements were made 1 hour after application. Niacinamide (4%) treatment for 1 h in low serum media (e.g. 0.2% FBS) activates the AMPK pathway to generate more energy in mature NHDF cells. Resveratrol salicylate (0.1%) treatment for 1 h specifically activates the AMPK pathway to generate more energy in both young and mature NHDF cells (red arrow). Surprisingly, the combination of niacinamide (4%) and resveratrol salicylate (0.1%) does not have an additive effect on AMPK activation. In fact, the level of activation was reduced.

Topically applied, compositions of the present invention enhance the production of energy in skin cells, and improve cell function. Oxidative damage associated with increased ATP production due to application of niacinamide is significantly reduced by resveratrol salicylate.

### Effects on Levels of AQP-3, CLDN-1 & CLDN-4 in Epidermal Keratinocytes

It has further been demonstrated that the combination of resveratrol salicylate and niacinamide regulates levels of AQP-3, CLDN-1 & CLDN-4 in epidermal keratinocytes, which provides opportunities to improve the barrier function of dry skin, leading to improvements in the moisture content of dry skin. The following experimental results demonstrate the usefulness of resveratrol salicylate in combination with niacinamide. Various measurements were made on 29 and 56 year old male, normal human epidermal keratinocytes (NHEKs).

Trehalose and betaine are osmolytes that help retain moisture, and ultimately support hydration of skin cells. We tested the effect of these osmolytes by themselves, and in conjunction with the combination of Niacinamide-Resveratrol salicylate. We performed the following gene expression studies in young and mature male NHEKs.

For each age group, twenty-five seed plates were prepared as follows. Normal human epidermal keratinocytes (75k/well in 1.875 mL of media) were allowed to culture for 24 hours under incubation at 37° C., 95% humidity, and 5% CO₂. The following treatments were prepared:
(1) Media (NHEK media supplemented with Keratinocytes Growth Media)
(2) DMSO (dimethyl sulfoxide) in media (259 μl DMSO to 6.741 ml of media)
(3) 0.2% trehalose in media
(4) 0.5% betaine in media
(5) 0.04% niacinamide+0.05% resveratrol salicylate in media
(6) In combination, 2 ml of each: 0.8% trehalose, 2% betaine, 0.16% niacinamide, 0.2% resveratrol salicylate.

Each well was treated with 1 ml of one of the foregoing treatments, and then incubated for 24 hours at 37° C., 95% humidity and 5% CO₂. Thereafter, cells were washed twice with 1 ml of PBS. Quantitative Real-Time PCR (qRT-PCR) was performed, as follows.

Isolation and the quantification of the RNA was performed with miRNeasy Micro Kit (Qiagen, Cat #217084), and Quant-it™ RiboGreen RNA Assay Kit (Invitrogen, Cat #R11490), respectively. One (1) μg of RNA was reverse transcribed into complementary DNA (cDNA) using a SuperScript™ IV VILO™ Master Mix (ThemoFisher, Cat #11756050) or TaqMan microRNA Reverse Transcription Kit (ThemoFisher, Cat #4366596).

The resulting cDNA was amplified using a Quant Studio 7 Flex system (Applied Biosystems) with a universal PCR master mix (Life Technologies, Cat #4440040) and the recommended PCR conditions for quantitative assessment of gene transcript levels in the samples.

To assess the expression of hydration targets genes, Aquaporin-3, Claudin-1 and Claudin-4, in 29y and 56y old NHEKs, Taqman assay probes CLDN-1 (Hs00221623_m1), CLDN-4 (Hs00976831_s1) and AQP-3 (Hs00185020_m1) were used.

The data were normalized to Glyceraldehyde-3-Phosphate Dehydrogenase control (GAPDH, Hs99999905_m1). qRT-PCR data were calculated using the 2-ΔΔCT method (Livak and Schmittgen, 2001).

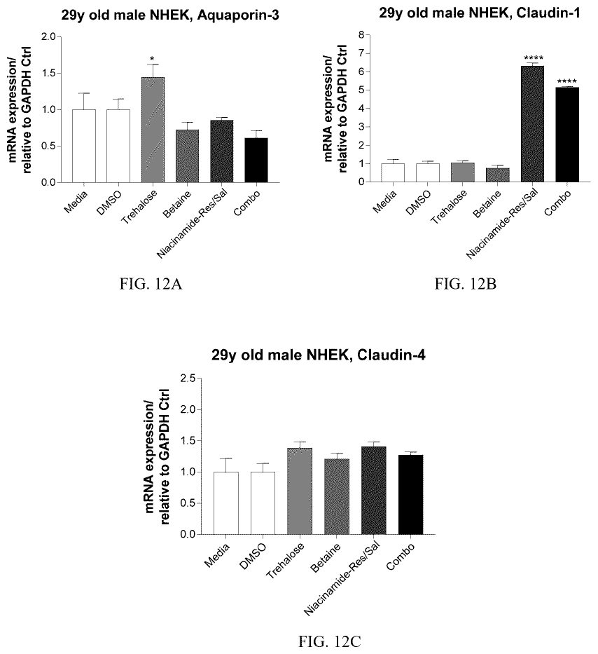

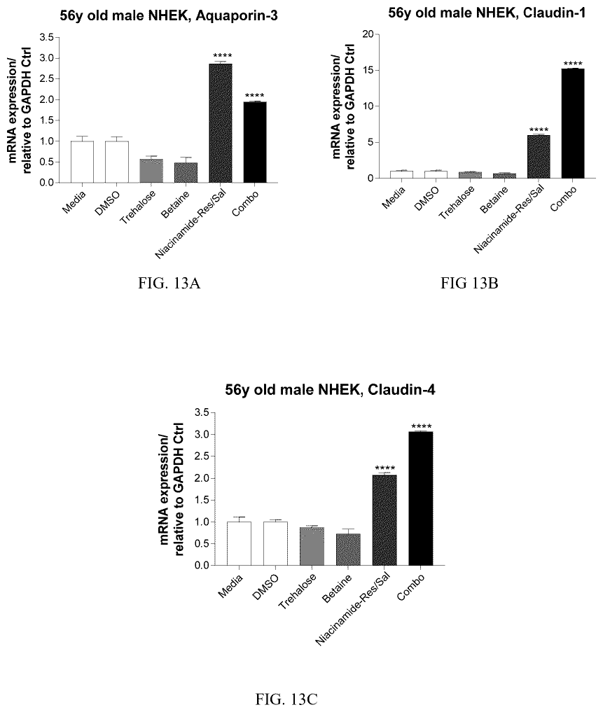

Data are representative of at least two independent biological replicates. Data were graphed and analyzed with the software GraphPad Prism (Prism 7.0). Statistical significance of differences between means was performed using 1-way ANOVA followed by Dunnett post-hoc test. The results are shown in FIGS. 12 and 13.

Referring to FIG. 12A, trehalose slightly increases AQP-3 gene expression levels in young keratinocytes cells, while all other test samples showed no increase.

Referring to FIG. 12B, trehalose and betaine had no effect on levels of CLDN-1 in young keratinocytes. The combination of niacinamide/resveratrol salicylate increases the levels of CLDN-1 in young keratinocytes in a statistically significant manner. The combination of trehalose/betaine/niacinamide/resveratrol salicylate also increases the levels of CLDN-1, which suggests that the combination of niacinamide/resveratrol salicylate is responsible for the increase.

Referring to FIG. 12C, all treatments produced only a slight increase CLDN-4 gene expression levels.

Referring to FIGS. 13A-C, trehalose and betaine have no significant effect on AQP-3, CLDN-1 or CLDN-4 gene expression. The combination of niacinamide/resveratrol salicylate increases the levels of AQP-3, CLDN-1 and CLDN-4 in mature keratinocytes in a statistically significant manner, especially AQP-3. The combination of trehalose/betaine/niacinamide/resveratrol salicylate also increases the levels of AQP-3, CLDN-1 and CLDN-4 in mature keratinocytes, especially CLDN-1 and CLDN-4, which is unexpected since trehalose and betaine by themselves had no, or slightly negative, effect.

Overall, the fact that the actives worked better on mature male NHEKs compared to the young ones is because the young cells already possess healthy basal levels of NMFs and osmolytes, which masks the beneficial effects of the active ingredients, whereas the mature cells, due to the aging, have lower basal levels of NMFs and osmolytes, so that the effect of the treatment is more pronounced.

Collectively, we have demonstrated that one or more resveratrol salicylate esters, when used in combination with niacinamide, mitigates the oxidative damage caused by increased ATP production in cells. Furthermore, the combination of niacinamide/resveratrol salicylate increases the levels of AQP-3, CLDN-1 and CLDN-4, especially AQP-3, while the combination of trehalose/betaine/niacinamide/resveratrol salicylate also increases the levels of AQP-3, CLDN-1 and CLDN-4, especially CLDN-1 and CLDN-4. Therefore, compositions according to the present invention are effective to increase the levels of hydration targets in mature cells, helping them to retain more moisture, and support a healthy hydrated skin.

In compositions according to the present invention, useful levels of niacinamide range from about 0.0001% to about 5% by total weight of the composition; for example about 0.001% to about 5%; for example about 0.01% to 3% by total weight of the composition. Preferably, the concentration of niacinamide is 2% to 4%. One or more resveratrol salicylates may be present ranging from about 0.001% to 5%, for example, from about 0.005% to 5%, or from about 0.01% to 5% by total weight of the composition. Preferably, the concentration of all resveratrol salicylates is about 0.5% to 2%. Some preferred embodiments of the invention comprise 0.1% to 2% trehalose and 0.1% to 2% betaine by total weight of the composition.

What is claimed is:

1. A topical composition comprising niacinamide and one or more resveratrol salicylates.

2. A topical composition according to claim 1 wherein the one or more resveratrol salicylate is selected from the group consisting of:
3-salicylate-5,4′-dihydroxystilbene;
5-salicylate-3,4′-dihydroxystilbene;
4′-salicylate-3,5-dihydroxystilbene;
3,5-disalicylate-4′-hydroxystilbene;
3,4′-disalicylate-5-hydroxystilbene;
4′,5-disalicylate-3-hydroxystilbene; and
3,5,4′-trisalicylate stilbene.

3. A topical composition according to claim 1 wherein the concentration of niacinamide ranges from about 0.0001% to about 5% by total weight of the composition, and the concentration of resveratrol salicylate ranges from about 0.001 to about 5% by total weight of the composition.

4. A topical composition according to claim 3 further comprising 0.1%-2% trehalose and 0.1%-2% betaine.

5. A topical composition according to claim 3 further comprising a cosmetically acceptable aqueous base, and optionally an oil phase.

6. A topical composition according to claim 5 wherein the composition is in the form of a water-in-oil or oil-in-water emulsion.
